(19) United States
(12) Patent Application Publication
(10) Pub. No.: US 2022/0238227 A1
(43) Pub. Date: Jul. 28, 2022

(54) MEDICAL INFORMATION PROCESSING APPARATUS, AND MEDICAL INFORMATION PROCESSING SYSTEM

(71) Applicant: Canon Medical Systems Corporation, Otawara-shi (JP)

(72) Inventors: Koji HIRATA, Kasukabe (JP); Katsuhiko FUJIMOTO, Saitama (JP); Kohei WATANABE, Utsunomiya (JP)

(73) Assignee: Canon Medical Systems Corporation, Otawara-shi (JP)

(21) Appl. No.: 17/578,827

(22) Filed: Jan. 19, 2022

(30) Foreign Application Priority Data
Jan. 28, 2021 (JP) 2021-012183

(51) Int. Cl. G16H 50/20; G16H 50/30; G16H 50/70; G16H 30/40; G16H 10/60

(52) U.S. Cl. CPC G16H 50/20; G16H 50/30; G16H 10/60; G16H 30/40; G16H 50/70

(57) ABSTRACT

According to one embodiment, a medical information processing apparatus includes processing circuitry. The processing circuitry predicts a progress relating to a disease that a patient contracts or may contract. The processing circuitry specifies a cell necessary for treating the disease. The processing circuitry predicts a first transition relating to a necessary amount of the cells and a second transition relating to a supply amount of the cells. The processing circuitry determines, based on the first transition and the second transition, a future time point when the necessary amount and the supply amount coincide, or when the supply amount is greater than the necessary amount.

### CROSS-REFERENCE TO RELATED APPLICATIONS

This application is based upon and claims the benefit of priority from Japanese Patent Application No. 2021-012183, filed Jan. 28, 2021, the entire contents of which are incorporated herein by reference.

### FIELD

Embodiments described herein relate generally to a medical information processing apparatus, and a medical information processing system.

### BACKGROUND

In recent years, with the increasing momentum for regenerative medicine, various problems in regard to actual clinical medicine have surfaced. One of the problems is a time lag from when a doctor examines a patient to when regenerative medicine is administered to the patient. In the regenerative medicine, after the doctor issues a prescription order of cells, based on the examination result of the patient, an institution that is entrusted with the prescription order cultures and processes cells, and provides the prepared cells to the doctor. In this process, since the culture and processing of cells require a time of several weeks to several months, a considerable length of time is needed until the cells are actually administered to the patient. Accordingly, the doctor is required to determine a prescription, such as the kind and amount of necessary cells, by predicting the progress relating to the stage, the severity level and the like of the patient's disease at a future time point when cells are administered to the patient.

However, since the above act of prescription depends on the experience and skill of an individual doctor, the act of prescription is a factor that hinders the uniformization of the quality of regenerative medicine. In addition, such a method is conceivable that many kinds and large amounts of cells for regenerative medicine are stored in advance in pharmaceutical companies or cell banks, so that the cells can immediately be provided from the pharmaceutical companies or cell banks in accordance with prescription orders. However, this method is not realistic from the viewpoint of the cost or the like relating to production and maintenance management of cells.

### BRIEF DESCRIPTION OF THE DRAWINGS

FIG. 1 is a view illustrating a configuration example of a medical information processing system according to an embodiment.

FIG. 2 is a view illustrating a configuration example of a medical information processing apparatus according to an embodiment.

FIG. 3 is a view illustrating an operation example of the medical information processing apparatus according to the embodiment.

FIG. 4 is a view illustrating an example of medical care information relating to a first patient.

FIG. 5 is a view illustrating an example of medical care information relating to a second patient.

FIG. 6 is a view illustrating an example of a transition relating to a necessary amount of cells.

FIG. 7 is a view illustrating an example of a transition relating to a supply amount of cells.

FIG. 8 is a view illustrating an example of transitions relating to a necessary amount of cells and a supply amount of cells.

FIG. 9 is a view illustrating an example of transitions relating to a necessary amount of cells and a supply amount of cells.

FIG. 10 is a view illustrating an example of an output result.

### DETAILED DESCRIPTION

In general, according to one embodiment, a medical information processing apparatus includes processing circuitry.

The processing circuitry predicts a first progress relating to a first disease that a patient contracts or may contract. The processing circuitry specifies a cell necessary for treating the first disease of the patient. The processing circuitry predicts a first transition relating to a necessary amount of the cells, based on the first progress. The processing circuitry predicts a second transition relating to a supply amount of the cells, based on information relating to a status of manufacture of the cells. The processing circuitry determines, based on the first transition and the second transition, a future time point when the necessary amount of the cells and the supply amount of the cells coincide, or when the supply amount of the cells is greater than the necessary amount of the cells. The processing circuitry outputs information relating to at least one of the first progress, the cell, the future time point, and the necessary amount of the cells at the future time point.

Hereinafter, a medical information processing apparatus and a medical information processing system according to embodiments will be described with reference to the accompanying drawings. In the embodiments below, it is assumed that parts denoted by like reference signs perform the same operation, and an overlapping description is omitted unless where necessary.

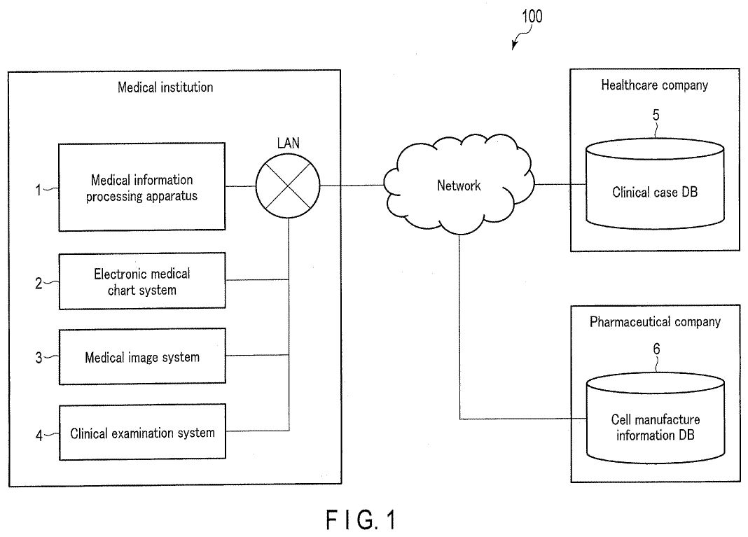

FIG. 1 is a view illustrating a configuration example of a medical information processing system 100 according to an embodiment.

The medical information processing system 100 is a system including a medical information processing apparatus 1, an electronic medical chart system 2, a medical image system 3, a clinical examination system 4, a clinical case DB 5, and a cell manufacture information DB 6. Of these components, the medical information processing apparatus 1, electronic medical chart system 2, medical image system 3 and clinical examination system 4 are installed in an identical medical institution, and are communicably connected to each other by a common LAN (Local Area Network). The clinical case DB 5 and cell manufacture information DB 6 are communicably connected to the LAN via a network. The clinical case DB 5 is installed, for example, in a healthcare company that manages the clinical case DB 5. The cell manufacture information DB 6 is installed in a pharmaceutical company that manages the cell manufacture information DB 6. Note that the communication between the LAN and the network may be executed via a proxy server that is installed between both networks. Note that the communication method between both networks may be of any kind, that is, may be a wired communication method or a wireless communication method.

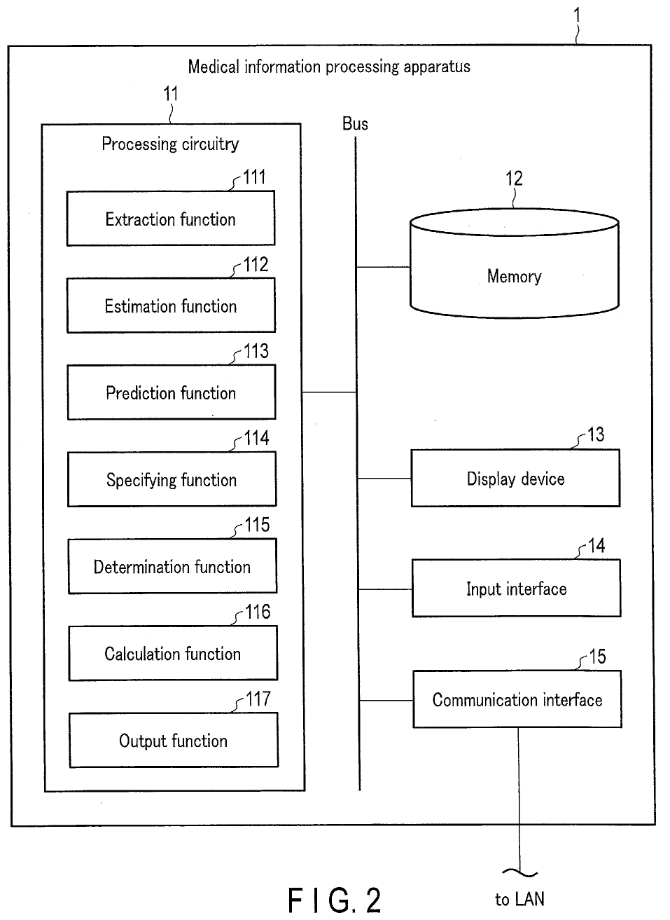

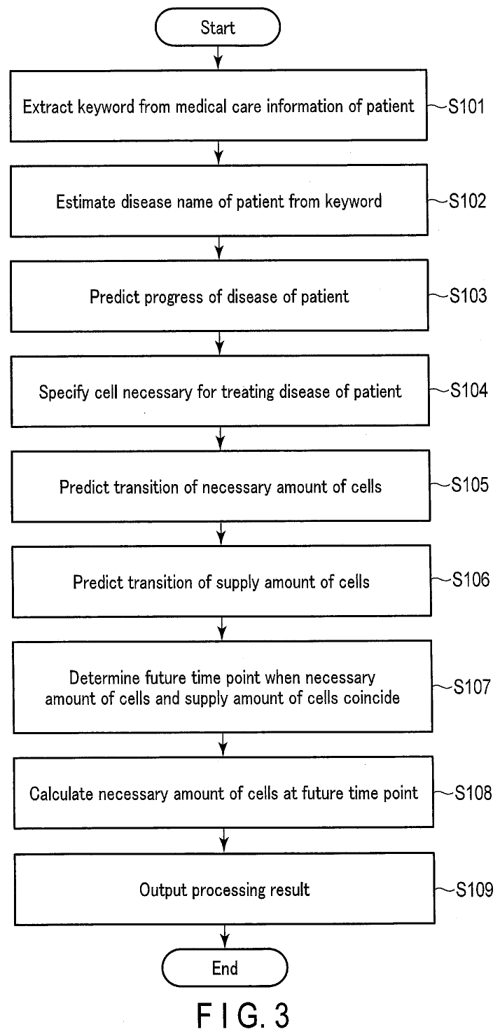

The medical information processing apparatus 1 is an apparatus serving as a functional center of the medical information processing system 100. The medical information processing apparatus 1 mutually transmits and receives data to and from the electronic medical chart system 2, medical image system 3, clinical examination system 4, clinical case DB 5 and cell manufacture information DB 6 via the LAN and the network, thereby executing various kinds of operations. A configuration example of the medical information processing apparatus 1 will be described later with reference to FIG. 2, and an operation example of the medical information processing apparatus 1 will be described later with reference to FIG. 3.

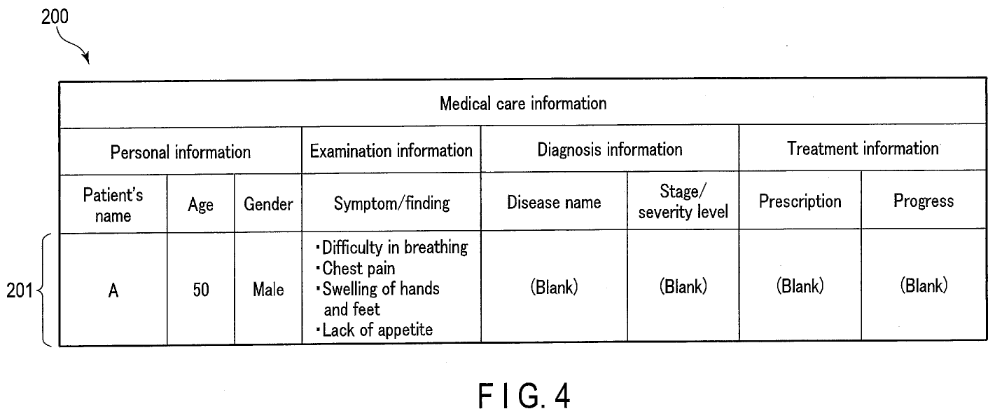

The electronic medical chart system 2 is a system that electronically records, stores and manages medical care information relating to various medical acts performed on a patient who visited a medical institution. In the present embodiment, it is assumed that the electronic medical chart system 2 includes examination images provided from the medical image system 3, and examination results provided from the clinical examination system 4. An example of a patient's medical care information stored in the electronic medical chart system 2 will be described later with reference to FIG. 4.

Note that it is assumed that the electronic medical chart system 2 includes medical care information of a patient (also referred to as “first patient”) who requires regenerative medicine using cells. It is assumed that the first patient has not yet been diagnosed or treated by a doctor, and that, at a time point of starting the operation illustrated in FIG. 3, which is executed by the medical information processing apparatus 1, the medical care information of the first patient includes personal information and examination information. The first patient is also referred to as “target patient”, “pre-treatment patient”, or “present patient”. In addition, it is assumed that the electronic medical chart system 2 stores at least one symptom relating to at least one first patient.

The medical image system 3 is a system that records, stores and manages various medical images relating to a patient who visited a medical institution. Medical images may be images of any kind, such as a CR (Computed Radiography) image, a CT (Computed Tomography) image, an MR (Magnetic Resonance) image, a UL (Ultrasonic) image, and an RI (Radio-Isotope) image. In the present embodiment, it is assumed that the medical image system 3 provides examination images to the electronic medical chart system 2. Note that the medical image system 3 is also referred to as PACS (Picture Archiving and Communication Systems).

The clinical examination system 4 is a system that records, stores and manages various examination results relating to a patient who visited a medical institution. The examination results include, for example, a result of a clinical examination (for example, a physiological examination, a specimen examination). In the present embodiment, it is assumed that the clinical examination system 4 provides examination results to the electronic medical chart system 2.

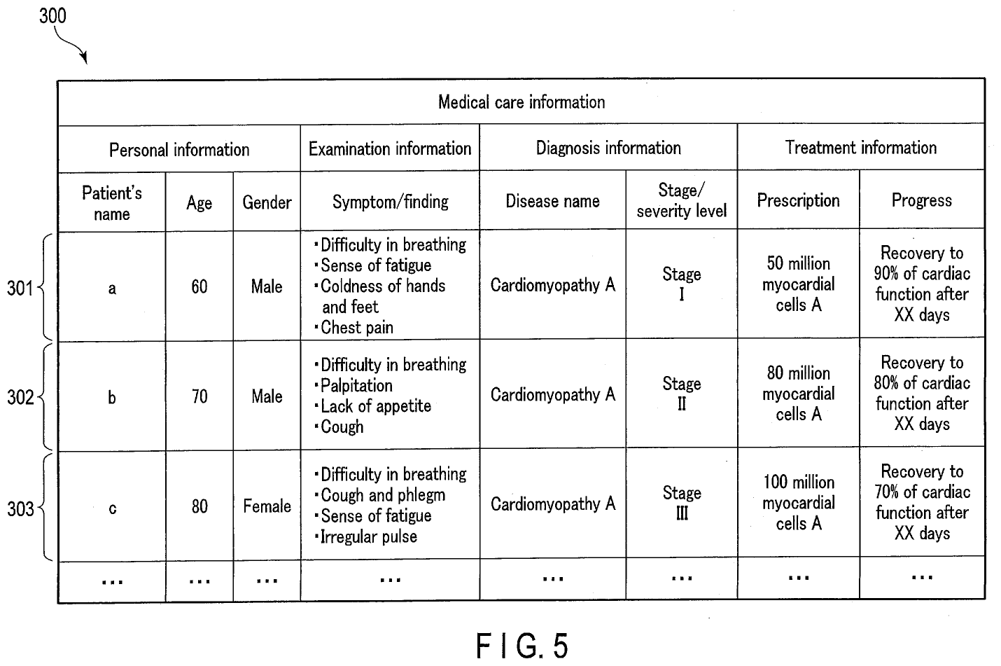

The clinical case DB 5 is a database that electronically stores medical care information relating to various medical acts performed on a patient. In the present embodiment, it is assumed that the medical care information stored in the clinical information DB 5 includes similar information to the medical care information stored in the electronic medical chart system 2. An example of a patient's medical care information stored in the clinical case DB 5 will be described later with reference to FIG. 5.

Note that it is assumed that the clinical case DB 5 includes medical care information of a patient (also referred to as “second patient”) who underwent regenerative medicine using cells. It is assumed that the second patient has been diagnosed and treated by a doctor, and that, at a time point of starting the operation illustrated in FIG. 3, which is executed by the medical information processing apparatus 1, the medical care information of the second patient includes diagnosis information and treatment information, in addition to the personal information and the examination information. The second patient is also referred to as “post-treatment patient”, or “past patient”. In addition, it is assumed that the clinical case DB 5 stores a plurality of cases (also referred to as “clinical cases”) relating to a plurality of second patients.

Note that the clinical case DB 5 may include not only clinical cases relating to the second patients, but also clinical cases relating to progresses of various diseases. The progress of a disease is, for example, information relating to variations of a diseased part and a symptom of a patient, which are involved in the progression of the disease. In addition, the progress of the disease may be information relating to variations of a diseased part and a symptom of the disease before and after treatment.

Note that the clinical case DB 5 may be replaced with a disease information DB including clinical cases from domestic and foreign medical DBs, general remarks, explanations and textbooks, or a treatment evidence DB including clinical cases from theses of domestic and foreign famous journals, and clinical reports. Specifically, as the clinical case DB 5, use can be made of a database containing clinical cases from various medical information sources, whether domestic or foreign. In addition, the clinical case DB 5 may include clinical cases relating to domestic and foreign clinical researches or clinical tests. The clinical cases relating to clinical researches or clinical tests may include, for example, cell names, names of executers, phases, kinds, periods, protocols, countries of execution, eligibility criteria, exclusion criteria, disease names, evaluation items and evaluation methods.

The cell manufacture information DB 6 is a database storing various kinds of information relating to cells and medicine (also referred to as “cell medicine”) using the cells. The cell manufacture information DB 6 includes, for example, information relating to the status of manufacture of cells, and the status of storing of cells. In addition, the cell manufacture information DB 6 may include cell names, product names, pharmaceutical company names, presence/absence in domestic markets, dosages, effects of treatment, side effects, and cautionary points relating to combined drugs/combined therapy. Specifically, the cell manufacture information DB 6 may include various kinds of information relating to cells and cell medicine, which pharmaceutical companies manufacture, and various kinds of information relating to the pharmaceutical companies.

FIG. 2 is a view illustrating a configuration example of the medical information processing apparatus 1 according to the embodiment.

The medical information processing apparatus 1 includes processing circuitry 11, a memory 12, a display device 13, an input interface 14, and a communication interface 15. The respective structural components are communicably connected to each other via a bus that is a common signal transmission path. Note that the respective structural components may not be implemented by individual hardware. For example, at least two of the structural components may be implemented by single hardware.

The processing circuitry 11 controls the operation of the medical information processing apparatus 1. The processing circuitry 11 includes, as hardware, a processor such as a CPU (Central Processing Unit), an MPU (Micro Processing Unit), or a GPU (Graphics Processing Unit). The processing circuitry 11 executes programs loaded in the memory 12 via the processor, thereby implementing functions (for example, an extraction function 111, an estimation function 112, a prediction function 113, a specifying function 114, a determination function 115, a calculation function 116, and an output function 117) corresponding to the programs. Note that each function may not be implemented by processing circuitry composed of a single processor. For example, each function may be implemented by processing circuitry composed by combining a plurality of processors. The processing circuitry 11 receives various kinds of data transmitted from, for example, the communication interface 15, and executes various information processes on the received data.

The extraction function 111 extracts a keyword from the medical care information of a patient.

The estimation function 112 estimates a disease name of the patient from the keyword.

The prediction function 113 predicts a first progress relating to a first disease that the patient contracts or may contract. In addition, the prediction function 113 predicts a first transition relating to a necessary amount of cells, based on the first progress. Furthermore, the prediction function 113 predicts a second transition relating to a supply amount of cells, based on the information relating to the status of manufacture of cells.

The specifying function 114 specifies a necessary cell for treating the first disease of the patient.

The determination function 115 determines, based on the first transition and the second transition, a future time point when the necessary amount of cells and the supply amount of cells coincide, or when the supply amount of cells is greater than the necessary amount of cells.

The calculation function 116 calculates a necessary amount of cells at the future time point.

The output function 117 outputs information relating to at least one of the first progress, the cell, the future time point, and the necessary amount of cells at the future time point.

The memory 12 stores information, such as data and programs, which the processing circuitry 11 uses. The memory 12 includes, as hardware, a semiconductor memory element such as a RAM (Random Access Memory). Note that the memory 12 may be a drive unit that reads and writes information from and to an external storage device such as a magnetic disc (a floppy (trademark) disc, a hard disk, a magneto-optical disc (MO), an optical disc (CD, DVD, Blu-ray (trademark)), a flash memory (a USB flash, a memory card, SSD), or magnetic tape. Note that a storage area of the memory 12 may be present in the inside of the medical information processing apparatus 1, or may be present in the external storage device.

The display device 13 displays information, such as data generated by the processing circuitry 11, or data stored in the memory 12. As the display device 13, use can be made of, for example, a cathode ray tube (CRT) display, a liquid crystal display (LCD), a plasma display, an organic EL (Electro-Luminescence) display (OELD), and a display of a tablet terminal. The display device 13 displays, for example, various processing results generated by the processing circuitry 11. Note that the display device 13 may be provided as a separate unit that can communicate with the medical information processing apparatus 1. At this time, the display device 13 may display a processing result that is output from the medical information processing apparatus 1.

The input interface 14 accepts an input from an operator, converts the accepted input to an electric signal, and outputs the electric signal to the processing circuitry 11. As the input interface 14, use cab be made of physical operating components, such as a mouse, a keyboard, a trackball, a switch, a button, a joystick, a touch pad, and a touch-panel display. Note that the input interface 14 may be a device that accepts an input from an external input device, which is separate from the medical information processing apparatus 1, converts the accepted input to an electric signal, and outputs the electric signal to the processing circuitry 11. The operator is, for example, a doctor who utilizes the medical information processing apparatus 1. For example, the doctor inputs, via the input interface 14, an operation of moving, minimizing, maximizing or closing a message box relating to various processing results displayed on the display device 13.

The communication interface 15 transmits and receives data to and from an external device. A freely chosen communication standard can be used between the communication interface 15 and the external device. For example, HL7 (Health Level 7) can be used for communications relating to medical character information, and DICOM (Digital Imaging and Communications in Medicine) can be used for communications relating to medical image information. The communication interface 15 is communicably connected to, for example, the LAN. In addition, the communication interface 15 acquires various kinds of data from the external device, and transmits the acquired data to the processing circuitry 11.

FIG. 3 is a view illustrating an operation example of the medical information processing apparatus 1 according to the embodiment.

It is assumed that, prior to the present operation example, a patient (first patient), who requires regenerative medicine using cells, visited a medical institution in which the medical information processing apparatus 1 is installed. For example, in the medical institution, the first patient writes, on a medical sheet, personal information including the name, age and gender of the patient, and information relating to the symptom of the patient. Next, the doctor examines the first patient while referring to the medical sheet, thereby acquiring the finding about the patient. The present operation example is started after the doctor inputs to the electronic medical chart system 2 the medical care information including the personal information relating to the first patient and the examination information relating to the symptom/finding of the first patient. Note that if the personal information and the examination information are already input to the electronic medical chart system 2, the doctor may execute the operation of the medical information processing apparatus 1 at a discretionary timing.

In step S101, the medical information processing apparatus 1 extracts a keyword from the patient's medical care information, by executing the extraction function 111. For example, the medical information processing apparatus 1 accesses the electronic medical chart system 2, thereby extracting a keyword from medical care information 200 of the first patient, which the doctor inputs to the electronic medical chart system 2. At this time, when examination information relating to a symptom or a finding of the first patient is included in the medical care information, the medical information processing apparatus 1 extracts a keyword relating to the first patient's disease from the examination information. For the extraction of the keyword, use may be made of a method, such as morphological analysis, which is used in general natural language processing. The extracted keyword is stored in the memory 12.

FIG. 4 is a view illustrating an example of the medical care information 200 relating to the first patient.

The medical care information includes personal information such as a patient's name, an age and a gender; examination information such as a symptom/finding; diagnosis information such as a disease name, and a stage/severity level; and treatment information such as a prescription and progress. The examination information includes various kinds of information obtained by a health care worker such as a doctor examining the patient. The diagnosis information includes various kinds of information obtained by a doctor diagnosing the patient's disease. The treatment information includes various kinds of information obtained by a medical care worker such as a doctor treating the patient's disease.

The “progress” included in the medical care information 200 means a progress of a disease. Specifically, the progress includes information relating to at least one of a diseased part and a symptom of the first patient, which are involved in the progression of the first patient's disease. In other words, it can be said that the progress is information relating to how the disease of the first patient has clinically progressed up to a time point when a predetermined number of days have passed. The progress includes, for example, a variation relating to the physical function of the patient after the prescription was administered.

The medical care information 200 includes a case 201 relating to the first patient that is the processing target of the medical information processing apparatus 1. Specifically, the case 201 includes “A” as the “patient's name”; “50” as the “age”; “male” as the “gender”; and “difficulty in breathing, chest pain, swelling of hands and feet, and lack of appetite” as the “symptom/finding”. In other words, the patient A is a male of 50 years old, and the symptoms and findings relating to the patient A include the difficulty in breathing, chest pain, swelling of hands and feet, and lack of appetite. Note that since the patient A has not yet been diagnosed or treated by the doctor, the items relating to the “diagnosis information” and “treatment information” are blank.

In step S101, the medical information processing apparatus 1 extracts, as the keyword relating to the disease of the patient A, at least one of the “difficulty in breathing, chest pain, swelling of hands and feet, and lack of appetite” from the “symptom/finding” of the patient A. It can also be said that the extracted keyword is a keyword characterizing the disease of the patient A.

In step S102, the medical information processing apparatus 1 estimates the disease name of the patient from the keyword, by executing the estimation function 112. For example, the medical information processing apparatus 1 searches the clinical case DB 5 by using the keyword extracted in step S101, thereby collecting clinical cases having a keyword identical or similar to the extracted keyword, among a plurality of clinical cases stored in the clinical case DB 5. Subsequently, the medical information processing apparatus 1 estimates that a disease name, which is correlated with the collected clinical cases, is the disease name of the first patient. At this time, a disease name correlated with the clinical case having a highest similarity to the extracted keyword, among the collected clinical cases, may be estimated to be the disease name of the first patient. The estimated disease name of the first patient is stored in the memory 12.

FIG. 5 is a view illustrating an example of medical care information 300 relating to a second patient.

FIG. 5 illustrates, among the cases included in the clinical case DB 5, a case 301, a case 302, a case 303, . . . , which have keywords identical of similar to the keywords “the difficulty in breathing, chest pain, swelling of hands and feet, and lack of appetite” extracted from the “symptom/finding” of the patient A.

The medical care information 300 includes the case 301, case 302, case 303, . . . , relating to patients (second patients) who underwent regenerative medicine using cells. Specifically, the case 301 includes “a” as the “patient's name”; “60” as the “age”; “male” as the “gender”; “difficulty in breathing, sense of fatigue, coldness in hands and feet, and chest pain” as the “symptom/finding”; “cardiomyopathy A” as the “disease name”; “stage I” as the “stage/severity level”; “50 million myocardial cells A” as “prescription”; and “recovery to 90% of cardiac function after XX days” as “progress”. In other words, the patient a is a male of 60 years old, and the symptoms or findings relating to the patient a include the difficulty in breathing, sense of fatigue, coldness in hands and feet, and chest pain. In addition, the patient a contracts cardiomyopathy A of stage I, and, as a result of the administration of 50 million myocardial cells A to the patient a, the cardiac function recovers to 90% of that at the normal time after XX days.

In step S102, since the “disease names” correlated with the case 301, case 302 and case 303 are all “cardiomyopathy A”, the medical information processing apparatus 1 estimates that the name of the disease of the patient A is “cardiomyopathy A”.

Note that the name of the disease of the first patient may be estimated, as described above, based on the clinical cases relating to the disease identical or similar to the disease of the first patient, but the process of estimating the name of the disease is not limited to the above. A similar process may be implemented as follows. A trained model, which is generated by training a network model used in machine learning represented by, for example, DNN (Deep Neural Network), by using training data that are a combination of input data, which are keywords relating to diseases in clinical cases, and correct answer data which are names of diseases in the clinical cases, is applied to the keyword relating to the disease of the first patient. An existing training method may be used as the training method of the network model using the training data. The trained model is stored in, for example, the memory 12.

For example, the medical information processing apparatus 1 creates training data that are a combination of input data, which are keywords “difficulty in breathing, sense of fatigue, coldness in hands and feet, and chest pain” relating to the disease in the case 301, and correct answer data that is “cardiomyopathy A”, which is the name of the disease in the case 301. As regards the case 302, case 303, . . . , the medical information processing apparatus 1 may generate trained models by creating many training data by similar methods and training network models. The medical information processing apparatus 1 applies, as the input data, the keywords “difficulty in breathing, chest pain, swelling of hands and feet, and lack of appetite”, which relate to the disease of the patient A in the case 201, to the trained model that was trained as described above, and thereby the trained model estimates that the name of the disease of the patient A is “cardiomyopathy A”.

Note that the estimation of the name of the disease of the first patient may be executed based on resident data including at least one of a disease tendency of residents, a population and an age distribution with respect to the area where the first patient resides. For example, if there is data indicating that the residents in the area where the first patient resides tend to contract pneumonia, the medical information processing apparatus 1 estimates that there is a high possibility that the first patient, too, contracts pneumonia. In addition, if there is data indicating that many elderly people live in the area where the first patient resides, the medical information processing apparatus 1 estimates that there is a high possibility that the first patient, too, contracts an age-related disease. As described above, the medical information processing apparatus 1 estimates the name of the disease which the first patient contracts or may contract.

In step S103, the medical information processing apparatus 1 predicts the process of the patient's disease by executing the prediction function 113. For example, the medical information processing apparatus 1 searches the clinical case DB 5 by using, as the keyword, the disease name of the first patient estimated in step S102, thereby collecting clinical cases having a disease name identical or similar to the keyword, among the clinical cases stored in the clinical case DB 5. Then, the medical information processing apparatus 1 predicts the progress of the disease of the first patient, based on the progress of the disease correlated with the collected clinical cases. The information relating to the predicted progress of the disease of the first patient is stored in the memory 12.

For example, based on the case 301, case 302 and case 303 illustrated in FIG. 5, the medical information processing apparatus 1 predicts the progress of “cardiomyopathy A” that was estimated as the disease name of the patient A illustrated in FIG. 4. The case 301 includes the “symptoms/findings” relating to the cardiomyopathy A of stage I, the case 302 includes the “symptoms/findings” relating to the cardiomyopathy A of stage II, and the case 303 includes the “symptoms/findings” relating to the cardiomyopathy A of stage III. In this manner, by learning the correspondence relation of each “stage/severity level” of the cardiomyopathy A to the “symptoms/findings” from the “examination information” and “diagnosis information” of the case 301, case 302 and case 303, the medical information processing apparatus 1 can predict the progress relating to the “symptoms/findings” of the cardiomyopathy A of the patient A. Here, the examination information is the information relating to the examination results at time points when the patients in the case 301, case 302 and case 303 were examined, and is, in other words, the information before each patient is treated. Accordingly, it can be said that the predicted progress is a progress relating to the “symptoms/findings” of the patient A before the cardiomyopathy A of the patient A is treated.

In addition, the case 301 includes the “prescription” and “progress” relating to the cardiomyopathy A of stage I, the case 302 includes the “prescription” and “progress” relating to the cardiomyopathy A of stage II, and the case 303 includes the “prescription” and “progress” relating to the cardiomyopathy A of stage III. In this manner, by learning the correspondence relation of each “stage/severity level” of the cardiomyopathy A to the “prescription” and “progress” from the “diagnosis information” and “treatment information” of the case 301, case 302 and case 303, the medical information processing apparatus 1 can predict the progress in the case where the prescription is administered to the cardiomyopathy A of the patient A. Here, the treatment information is the information relating to the treatment results after the patients in the case 301, case 302 and case 303 underwent treatment, and is, in other words, the information after each patient is treated. Accordingly, it can be said that the predicted progress is a progress relating to the functional prognosis of the patient A after the cardiomyopathy A of the patient A is treated.

Note that, although not illustrated in FIG. 4 and FIG. 5, when an “examination image” is included as the examination information, the medical information processing apparatus 1 can predict, by the same operation as described above, the progress relating to the “examination image” in each “stage/severity level” of the disease. It can also be said that this progress is a progress before the disease is treated. In addition, when an “examination result” is included as the examination information, the medical information processing apparatus 1 can predict, by the same operation as described above, the progress relating to the “examination result” in each “stage/severity level” of the disease. It can also be said that this progress is a progress before the disease is treated.

Besides, note that, although not illustrated in FIG. 4 and FIG. 5, when an “examination image” is included as the treatment information, the medical information processing apparatus 1 can predict, by the same operation as described above, the progress relating to the “examination image” in each “stage/severity level” of the disease. It can also be said that this progress is a progress after the disease is treated. In addition, when an “examination result” is included as the treatment information, the medical information processing apparatus 1 can predict, by the same operation as described above, the progress relating to the “inspection result” in each “stage/severity level” of the disease. It can also be said that this progress is a progress after the disease is treated.

Note that the progress relating to the disease of the first patient may be estimated, as described above, based on the clinical cases relating to the disease identical or similar to the disease of the first patient, but the process of estimating the progress is not limited to the above. A similar process may be implemented as follows. A trained model, which is trained by using training data that are a combination of input data, which are names of diseases in clinical cases, and correct answer data which are progresses relating to diseases in the clinical cases, is applied to the name of the disease of the first patient. The trained model is stored in, for example, the memory 12.

In step S104, the medical information processing apparatus 1 specifies a cell that is necessary for treating the disease of the patient, by executing the specifying function 114. For example, the medical information processing apparatus 1 specifies the cell used as a treatment method for the disease in the clinical cases, which were collected in step S102 or step S103, as the cell that is necessary for treating the disease of the first patient. The information relating to the specified cell that is necessary for treating the disease of the first patient is stored in the memory 12.

For example, the medical information processing apparatus 1 specifies that the “myocardial cell A” is necessary for treating the patient A, from the prescription “50 million myocardial cells A” of the patient a in the case 301 illustrated in FIG. 5.

In step S105, the medical information processing apparatus 1 predicts the transition of the necessary amount of cells, by executing the prediction function 113. For example, the medical information processing apparatus 1 predicts the necessary amount of cells, which is necessary for the treatment of the first patient, based on the clinical cases relating to the disease identical or similar to the disease of the first patient.

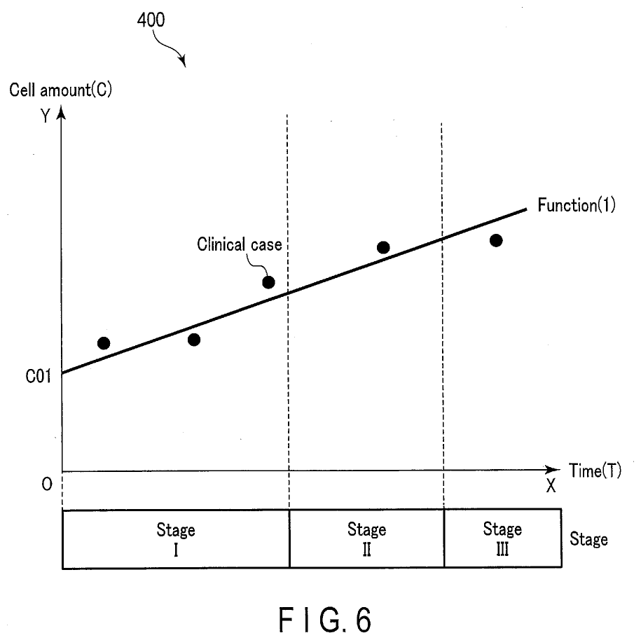

FIG. 6 is a view illustrating an example of a transition relating to a necessary amount of cells.

A necessary amount transition graph 400 illustrated in FIG. 6 is a graph schematically illustrating the transition of the necessary amount of cells with the passing of time. Specifically, the transition of the necessary amount of cells in a two-dimensional coordinate plane, in which the abscissa axis (X axis) is time (T) and the ordinate axis (Y axis) is a cell amount (C), is represented by a function (1). In addition, under the abscissa axis, the stage of the disease with the passing of time (T) is expressed by the same time scale as the time (T). Here, such a state is illustrated that the stage of the disease transitions from a mild stage to a severe stage, like stage I, stage II, stage III, . . . , with the passing of time (T) from the origin O as the starting point.

The function (1) is expressed by a linear line extending in an upper-right direction, with a cell amount C01 being located at a Y intercept. The reason for this is that the necessary cell amount (C) for the treatment of the disease is generally considered to increase with the progression of the disease. Needless to say, the function (1) is not limited to the linear line, and may be expressed by a curve such as a quadratic curve. Note that the function (1) may be any function by which the cell amount (C) at a given time point is uniquely determined. The function (1) is a function with time (T) as a variable.

For example, the function (1) is derived in the following manner.

To begin with, based on the clinical cases included in the clinical case DB 5, the medical information processing apparatus 1 plots coordinates corresponding to the clinical cases on the two-dimensional coordinate plane. Specifically, the medical information processing apparatus 1 plots coordinates (X, Y), with the “stage/severity level” of the disease in each clinical case being set on the X coordinate, and with the “prescribed cell amount” in each clinical case being set on the Y coordinate. Then, the medical information processing apparatus 1 derives the function (1) that precisely reproduces the plotted coordinates. At this time, the function (1) may be calculated in such a manner as to minimize the error between the value of the function (1) and the value of the Y coordinate of each coordinates (least squares method). Specifically, in accordance with the distribution of coordinates, approximation may be executed by a freely chosen regression analysis (for example, simple linear regression analysis, multiple linear regression analysis).

Note that although the transition relating to the necessary cell amount for treating the first patient's disease may be estimated, as described above, based on the clinical cases relating to the disease identical or similar to the first patient's disease, the process of estimating the transition is not limited to the above. A similar process may be implemented as follows. A trained model, which is trained by using training data that are a combination of input data, which are transitions of diseases in clinical cases, and correct answer data which are prescription amounts of cells relating to diseases in the clinical cases, is applied to the transition of the disease of the first patient, which is predicted in step S103. The trained model is stored in, for example, the memory 12.

In step S106, the medical information processing apparatus 1 predicts the transition of the supply amount of cells, by executing the prediction function 113. For example, the medical information processing apparatus 1 predicts the transition relating to the supply amount of cells, based on information relating to the status of manufacture of cells.

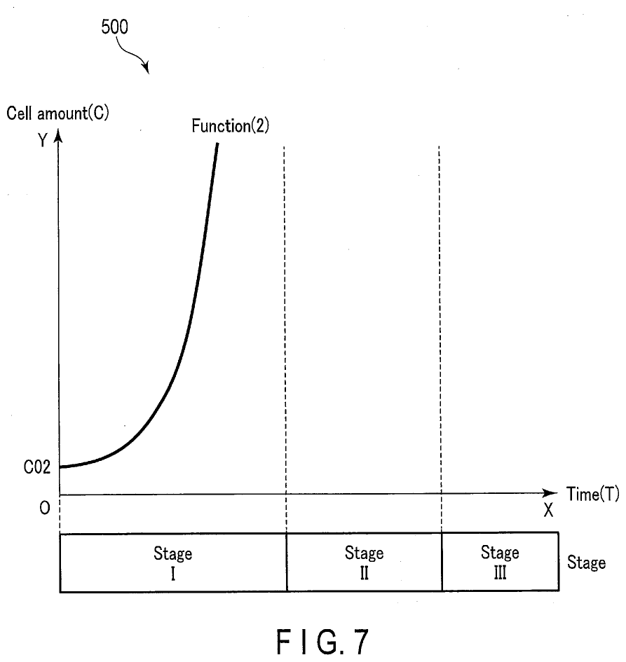

FIG. 7 is a view illustrating an example of a transition relating to the supply amount of cells.

A supply amount transition graph 500 illustrated in FIG. 7 is a graph schematically illustrating the transition of the supply amount of cells with the passing of time. Specifically, the transition of the supply amount of cells in a two-dimensional coordinate plane, in which the abscissa axis (X axis) is time (T) and the ordinate axis (Y axis) is a cell amount (C), is represented by a function (2). In addition, under the abscissa axis, the stage of the disease with the passing of time (T) is expressed by the same time scale as the time (T). Here, such a state is illustrated that the stage of the disease transitions from a mild stage to a severe stage, like stage I, stage II, stage III, . . . , with the passing of time (T) from the origin O as the starting point. Note that the time scale of the time axis illustrated in FIG. 6 is identical to the time scale of the time axis illustrated in FIG. 7.

The function (2) is expressed by a curve extending in an upper-right direction, with a cell amount C02 being located at a Y intercept. Specifically, the function (2) indicates the variation of the cell amount (C) in the case where the culture of cells is started from the predetermined cell amount C02 and the cells proliferate exponentially. Needless to say, the function (2) is not limited to the curve, and may be expressed by a linear line. Note that the function (2) may be any function by which the cell amount (C) at a given time point is uniquely determined. The function (2) is a function with time (T) as a variable.

For example, the function (2) is derived in the following manner.

To begin with, the medical information processing apparatus 1 accesses the cell manufacture information DB 6, thereby acquiring information relating to the status of manufacture of a manufacture line of cells. Based on the acquired information, the medical information processing apparatus 1 may predict the transition of the supply amount of cells with the passing of time. The method of predicting the transition of the supply amount may be a general calculation method, for example, a calculation method in which an exponential increase is assumed from a cell amount at the time of starting cell culture and a time needed for one-time cell division.

Note that, for the purpose of convenience of description, the function (2) is described on the assumption that the proliferation curve of cells corresponds to the transition of the supply amount of cells, but the function (2) is not limited to this. In fact, various processes, such as processing of cells and transportation of cells, are included during the process from the start of manufacture of cells until the cells are administered to the patient that requires regenerative medicine. The medical information processing apparatus 1 may derive the function (2) in consideration of the time needed for these processes. For example, by taking into account the loss of cells at the time of processing, the function (2) may be derived with such a margin that the supply amount of cells becomes greater than the necessary amount of cells administered to the patient.

In step S107, the medical information processing apparatus 1 executes the determination function 115, thereby determining a future time point when the necessary amount of cells and the supply amount of cells coincide, or when the supply amount of cells is greater than the necessary amount of cells. Specifically, the medical information processing apparatus 1 determines a future time point when the necessary amount of cells and the supply amount of cells coincide, or when the supply amount of cells is greater than the necessary amount of cells, based on the transition of the necessary amount of cells predicted in step S105 and the transition of the supply amount of cells predicted in step S106. Hereinafter, an example will be described in which the future time point is the time point when the necessary amount of cells and the supply amount of cells coincide.

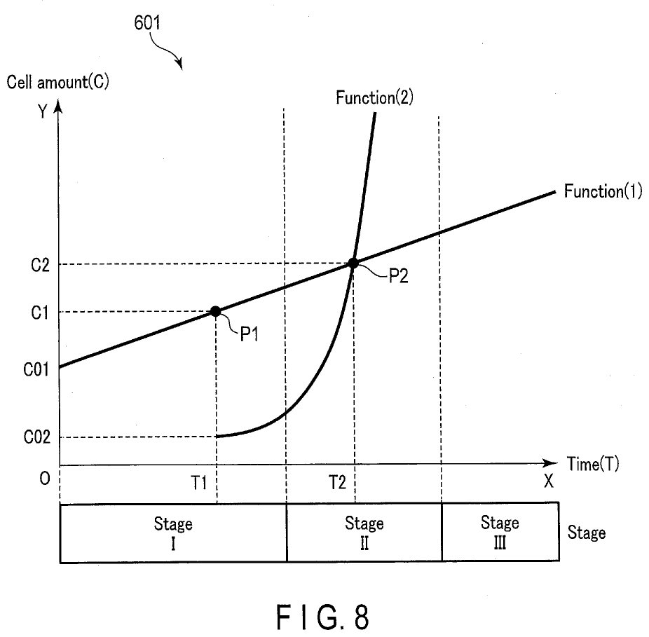

FIG. 8 is a view illustrating an example of transitions relating to a necessary amount of cells and a supply amount of cells.

A composite graph 601 illustrated in FIG. 8 is a graph in which the graph of the function (1) illustrated in FIG. 6 and the graph of the function (2) illustrated in FIG. 7 are composited on an identical two-dimensional coordinate plane. As described above, since the time scale of FIG. 6 and the time scale of FIG. 7 are identical, the graph of the function (1) and the graph of the function (2) are composited and displayed on the identical two-dimensional coordinate plane, without being enlarged or reduced. Note that the start point of the function (2) is shifted to a time point T1 in the X-axis direction in accordance with the stage of the disease of the first patient. Here, a case is assumed in which the manufacture of cells was started at the same time point as when the first patient visited the medical institution.

Time point T1 is the time point when the first patient visited the medical institution, and is the time point when the manufacture of cells necessary for the treatment of the first patient was started. It is assumed that the cell amount at an intersection P1 of the function (1) at time point T1 is C1. At this time, it can be said that the cell amount C1 is a necessary cell amount for treating the first patient's disease at the time point when the first patient visited the medical institution. Here, the cell amount C1 is greater than the cell amount C02 that is the supply amount of cells at the same time point.

Time point T2 is a time point at an intersection P1 between the function (1) and the function (2). The intersection P2 is a time point at which the value of the function (1) and the value of the function (2) coincide. It is assumed that the cell amount of the function (1) at time point T2 is C2. At this time, it can be said that the cell amount C2 is a cell amount at a time point when the necessary amount of cells and the supply amount of cells coincide. In other words, the time point T2 is a time point when the treatment of the first patient can be started.

In addition, it can be said that a period from time point T1 to time point T2 is a standby period until the treatment. Furthermore, by observing the stage of the disease at time point T2, the stage of the disease of the patient after the passage of the standby period can be understood. At this time, by searching the clinical case DB 5 by using the stage of the disease at time point T2 as the keyword, the medical information processing apparatus 1 can also predict the symptom/finding or the like of the patient after the passage of the standby period. Besides, it can be said that the cell amount C2 at time point T2 is the necessary cell amount at the time of starting the treatment after the passage of the standby period.

Specifically, by taking into account both the increase of the necessary cell amount for treatment with the progression of the patient's disease and the increase of the supplied cell amount with the passing of time, the medical information processing apparatus 1 predicts the future time point when the necessary cell amount and the supplied cell amount are balanced. As described above, since the supply of cells requires a predetermined time, it is considered that the patient's disease becomes worse and the necessary cell amount for treatment increases. Here, since the cell amount C02 at the cell supply start time point T1 is less than the necessary cell amount C1 for treating the patient at the same time point, it can be said that the rate of the cell supply controls the timing of the start of treatment. It can be said that, by taking into account both the rate of increase of the necessary cell amount for treatment and the rate of increase of the supplied cell amount, the medical information processing apparatus 1 calculates time point T2 that is appropriate for starting the treatment of the patient's disease.

Note that since the supply amount of cells is greater than the necessary amount of cells after time point T2, a freely chosen time point after time point T2 may be set as the future time point (the time point of starting treatment). The necessary amount of cells at this time point can be calculated by substituting time (T) at this time point in the function (1). From this viewpoint, it can be said that time point T2 is an earliest time point at which treatment can be started.

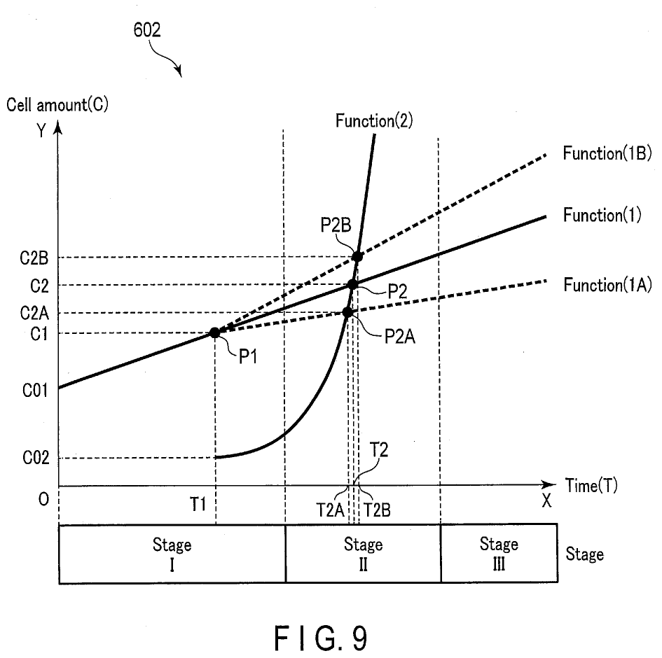

Note that the graph of the function (1) indicates the transition of the general necessary amount of cells calculated from past clinical cases. Here, as illustrated in FIG. 9, there may be a function (1A) in the case where the disease of the first patient transitions in a better condition from time point T1 than in the transition illustrated in the graph of the function (1), and a function (1B) in the case where the disease of the first patient transitions in a worse condition from time point T1 than in the transition illustrated in the graph of the function (1). The function (1A) and the function (1B) are graphs relating to the transition of the necessary amount of cells.

In a composite graph 602 illustrated in FIG. 9, it is assumed that an intersection between the function (1A) and the function (2) is P2A, and an intersection between the function (1B) and the function (2) is P2B. Further, it is assumed that a time point at the intersection P2A is T2A, a cell amount at the intersection P2A is C2A, a time point at the intersection P2B is T2B, and a cell amount at the intersection P2B is C2B. As illustrated in FIG. 9, depending on whether the transition of the patient's disease is better or worse, it is predicted that the time point when the treatment can be performed shifts before or after T2 (T2A-T2B). Moreover, it can be predicted that the necessary cell amount for starting treatment shifts before or after C2 (C2A-C2B). In this manner, by predicting the transition of the necessary cell amount for treatment by a plurality of graphs, the medical information processing apparatus 1 can predict the timing of starting treatment or the necessary cell amount for starting treatment in the case where the patient's disease transitions in a better condition or a worse condition.

In step S108, the medical information processing apparatus 1 calculates a necessary cell amount at the future time point determined in step S107, by executing the calculation function 116. This calculation may be executed from the graph (function (1)) relating to the necessary amount of cells.

In FIG. 8, the cell amount C2 at time point T2 corresponds to the necessary amount of cells at the future time point. In other words, the cell amount C2 at time point T2 is the necessary cell amount at the time of starting treatment after the passage of the standby time. Since the time point T2 is the time point when the value of the function (1) and the value of the function (2) coincide, the cell amount C2 may be calculated by substituting the time point T2 in either the function (1) or the function (2).

In step S109, the medical information processing apparatus 1 outputs a processing result by executing the output function 117. Specifically, the medical information processing apparatus 1 outputs to the display device 13 at least one of the processing results obtained in the processes of step S101 to step S108. The processing results include, for example, the disease name of the first patient, which is estimated in step S102; the progress of the first patient's disease, which is predicted in step S103; the necessary cell for treating the first patient's disease, which is specified in step S104; the future time point determined in step S107, at which the necessary amount of cells and the supply amount of cells coincide; and the necessary amount of cells at the future time point, which is calculated in step S108. The medical information processing apparatus 1 integrates the processing results stored in the memory 12, converts the integrated processing result into a predetermined format (for example, text), and outputs the converted processing result to the display device 13.

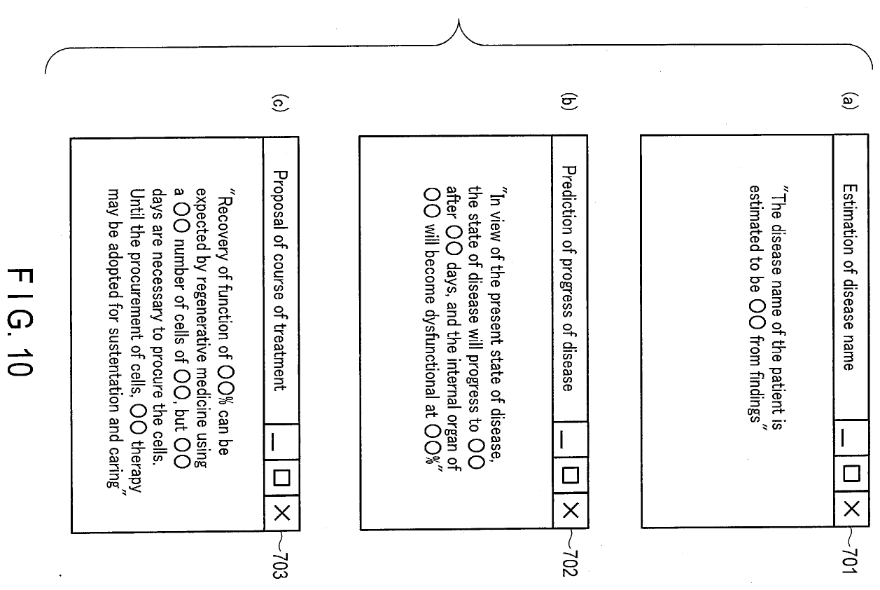

FIG. 10 is a view illustrating an example of an output result.

In FIG. 10, processing results are displayed by a plurality of message boxes. Specifically, FIG. 10 includes a message box 701 (FIG. 10 (a)), a message box 702 (FIG. 10 (b)), and a message box 703 (FIG. 10 (c)). Each message box includes a title representing the kind of each processing result, and a text representing the content of each processing result. The respective message boxes may be displayed in parallel or in a superimposed manner on the screen of the display device 13.

The message box 701 represents the disease name of the first patient, which is estimated in step S102. The message box 701 displays “Estimation of disease name” as the title, and displays “The disease name of the patient is estimated to be ∘∘ from findings” as the text expressing the processing result relating to “Estimation of disease name”. For example, when the first patient is the patient A illustrated in FIG. 4, the message box 701 displays such a message as “The disease name of patient. A is estimated to be cardiomyopathy A from findings”.

The message box 702 displays the progress of the disease of the first patient, which is predicted in step S103. The message box 702 displays “Prediction of progress of disease” as the title, and displays “In view of the present state of disease, the state of disease will progress to ∘∘ after 00 days, and the internal organ of ∘∘ will become dysfunctional at ∘∘ %” as the text expressing the processing result relating to “Prediction of progress of disease”. For example, when the first patient is the patient A illustrated in FIG. 4, the message box 702 displays such a message as “The state of disease will progress to stage II after XX days, and the heart will become dysfunctional at 20%”.

The message box 703 indicates the necessary cell for treating the first patient's disease, which is specified in step S104, the future time point determined in step S107, at which the necessary amount of cells and the supply amount of cells coincide, and the necessary amount of cells at the future time point, which is calculated in step S108. The message box 703 displays “Proposal of course of treatment” as the title, and displays “Recovery of function of ∘∘ % can be expected by regenerative medicine using a ∘∘ number of cells of ∘∘, but ∘∘ days are necessary to procure the cells. Until the procurement of cells, ∘∘ therapy may be adopted for sustentation and caring” as the text expressing the processing result relating to “Proposal of course of treatment”. For example, when the first patient is the patient A illustrated in FIG. 4, the message box 703 displays such a message as “Recovery of function of 90% can be expected by regenerative medicine using 50 million myocardial cells A, but 60 days are necessary to procure the cells. Until the procurement of cells, XX therapy may be adopted for sustentation and caring”.

Note that the contents of the texts displayed in the message boxes are not limited to the above. For example, in the case of the message box 701, any kind of message, which is the text indicating the “disease name of the first patient”, can be used. In the case of the message box 702, any kind of message, which is the text indicating the “progress of the disease of the first patient”, can be used, and any information, which is stored in the clinical case DB 5 and is used in the calculation of the “prediction of the progress of the disease”, may be displayed. In the case of the message box 703, any message having the content, which indicates the “necessary cell for treating the first patient's disease”, “future time point at which the necessary amount of cells and the supply amount of cells coincide” and “necessary amount of cells at the future time point”, can be used, and any information, which is stored in the clinical case DB 5 and is used in the calculation of the “proposal of the course of treatment”, may be displayed.

The following advantageous effects can be obtained by the doctor confirming, on the display device 13, the processing results which the medical information processing apparatus 1 outputs. For example, the doctor can estimate the disease name of the patient by confirming the message box 701. Specifically, the medical information processing apparatus 1 can support the doctor in diagnosing the disease name of the patient. In addition, the doctor can predict the progress of the patient's disease by confirming the message box 702. Specifically, the medical information processing apparatus 1 can support the doctor in predicting the progress of the patient's disease. Besides, the doctor can decide on the course of treatment of the patient by confirming the message box 703. Specifically, the medical information processing apparatus 1 can support the doctor in deciding on the course of treatment of the patient.

Note that the output mode of the processing results is not limited to the message boxes. For example, the processing results may be output by images or voice. In addition, the contents of the message box 701, message box 702 and message box 703 may be output by voice. Moreover, the graphs illustrated in FIG. 8 and FIG. 9 may be displayed on the display device 13.

Note that the medical information processing apparatus 1 may output a prescription order to manufacture an amount of cells, which corresponds to the calculated necessary amount of cells at a future time point, to an institution that manufactures cells. Thereby, the load on the doctor is decreased, since the medical information processing apparatus 1 automatically issues the prescription order, without the doctor manually issuing the prescription order.

Note that the medical information processing apparatus 1 may extract various kinds of information relating to cells for regenerative medicine from the cell manufacture information DB 6, and may cause the display device 13 to display the extracted information as the processing result. Specifically, the display device 13 may display, in addition to the name of the cell for regenerative medicine, the product name, presence/absence in domestic markets, dosage, effects of treatment, side effects, cautionary points of combined drugs/combined therapy, and the period needed until procurement.

In the above operation example, the process may be executed by a trained model which can execute each step, but the operation example is not limited to this. For example, there may be a trained model which can execute a series of operations of steps S101 to S109. This trained model may search the clinical case DB 5, based on the medical care information of the patient stored in the electronic medical chart system 2, and may execute the diagnosis prediction and the progress prediction of the patient's disease, based on clinical cases similar to the case of the patient.

The operation example of the medical information processing apparatus 1 according to the embodiment has been described above. Note that the order of steps S101 to S109 is not limited to this. For example, the “specifying of the cell necessary for treating the patient's disease” of step S104 may be executed before the “prediction of the progress of the patient's disease” of step S103. In addition, the “prediction of the transition of the supply amount of cells” of step S106 may be executed before the “prediction of transition of the necessary amount of cells” of step S105. Besides, the processing results may not be output batchwise in step S109. Each time a processing result is calculated in each step, the processing result may be output to the display device 13.

Note that the above operation is applicable to any disease, if the disease require regenerative medicine using cells. Hereinafter, examples of the medical information processing apparatus 1 will be describing by taking myocardial regenerative medicine and CAR-T regenerative medicine as examples of regenerative medicine using cells. It is assumed that the disease that is a treatment target in the myocardial regenerative medicine is cardiomyopathy, and the disease that is a treatment target in the CAR-T regenerative medicine is hematological malignancy (for example, B-cell leukemia, lymphoma).

### Example 1: Myocardial Regenerative Medicine

Cardiomyopathy is a general term of diseases in which the cardiac function progressively deteriorates due to the abnormality of the cardiac muscle. The cardiomyopathy is induced by, for example, cardiotoxicity of an anticancer drug (for example, anthracycline anticancer drug, monoclonal antibody anticancer drug) in cancer chemical therapy. Pathologically, in the cardiomyopathy, necrosis and vacuolar degeneration of myocardial cells, and misarrangement and deficiency of myocardial fibers are observed. Here, it is assumed that a patient (also referred to as “patient B”), who is suspected to contract cardiomyopathy, visited a medical institution in which the medical information processing apparatus 1 is installed, and medical care information including the symptom/finding, examination image and examination result of the patient was recorded in the electronic medical chart system 2, based on the result of examination of the patient by a health care worker, such as a doctor. The doctor monitors the progression of the cardiomyopathy of the patient by external diagnosis or image diagnosis, and, when the doctor judges that regenerative medicine is necessary for the patient, the operation by the medical information processing apparatus 1 is started.

The medical information processing apparatus 1 executes the operation (steps S101 to S109) illustrated in FIG. 3, based on the keyword relating to the symptom/finding of cardiomyopathy which was input to the electronic medical chart system 2, the image diagnosis data that is the examination image, and the external diagnosis data that is the examination result. Thereby, the medical information processing apparatus 1 can predict the necessary time, kind and necessary amount of cells for transplantation, which are necessary for treating the patient B, and can exemplarily show them to the doctor. Moreover, the doctor can decide on the prescription for the patient B, based on the proposal relating to the course of treatment, which is exemplarily shown by the medical information processing apparatus 1.

### Example 2: CAR-T Regenerative Medicine

CAR-T (chimeric antigen receptor T-cell) regenerative medicine is a cell therapeutic method in which T-cells sampled from the patient are genetically modified to produce CARs (chimeric antigen receptors) that recognize the antigen of cancer cells that are a target, and the T-cells are brought back into the patient's body, thereby causing the T-cells to attack and kill the cancer cells. Here, it is assumed that a patient (also referred to as “patient C”), who is suspected to contract hematological malignancy, visited a medical institution in which the medical information processing apparatus 1 is installed, and medical care information including the symptom/finding and examination result of the patient was recorded in the electronic medical chart system 2, based on the result of the examination of the patient by a health care worker, such as a doctor. The doctor monitors the progression of the disease of the patient by external diagnosis, and, when the doctor judges that regenerative medicine is necessary for the patient, the operation by the medical information processing apparatus 1 is started.

The medical information processing apparatus 1 executes the operation (steps S101 to S109) illustrated in FIG. 3, based on the keyword relating to the symptom/finding of hematological malignancy which was input to the electronic medical chart system 2, and the external diagnosis data that is the examination result. Thereby, the medical information processing apparatus 1 can predict the necessary time, kind and necessary amount of cells for transplantation, which are necessary for treating the patient C, and can exemplarily show them to the doctor. Moreover, the doctor can decide on the prescription for the patient C, based on the proposal relating to the course of treatment, which is exemplarily shown by the medical information processing apparatus 1.

Note that the medical information processing apparatus 1 according to the present embodiment is applicable to not only auto-CAR-T regenerative medicine which utilizes T-cells of a patient himself/herself, but also allo-CAR-T regenerative medicine using T-cells derived from a third person other than the patient. In the allo-CAR-T regenerative medicine, T-cells prepared from an iPS cell (Induced Pluripotent Stem cell) or an ES cell (Embryonic Stem cell) may be utilized.

According to at least one of the above-described embodiments, a prescription of cells by a doctor can be supported in regenerative medicine. In addition, a future prediction by a doctor in regard to the stage of disease and the severity level of a patient can be supported. Furthermore, a prescription of cells for regenerative medicine by a doctor can be supported. Besides, the quality of regenerative medicine can be uniformized without depending on the skill and experience of an individual doctor. Thereby, the quality of the regenerative medicine can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising:
processing circuitry configured to:
predict a first progress relating to a first disease that a patient contracts or may contract;
specify a cell necessary for treating the first disease of the patient;
predict a first transition relating to a necessary amount of the cells, based on the first progress;
predict a second transition relating to a supply amount of the cells, based on information relating to a status of manufacture of the cells;
determine, based on the first transition and the second transition, a future time point when the necessary amount of the cells and the supply amount of the cells coincide, or when the supply amount of the cells is greater than the necessary amount of the cells; and
output information relating to at least one of the first progress, the cell, the future time point, and the necessary amount of the cells at the future time point.

2. The medical information processing apparatus of claim 1, wherein the processing circuitry predicts the first progress, based on a clinical case relating to a second disease identical or similar to the first disease of the patient.

3. The medical information processing apparatus of claim 2, wherein the processing circuitry estimates the first progress by applying a trained model, which is trained by using training data that is a combination of input data, which is a name of the second disease in the clinical case, and correct answer data which is a second progress relating to the second disease in the clinical case, to a name of the first disease.

4. The medical information processing apparatus of claim 1, wherein the processing circuitry predicts the first transition, based on a clinical case relating to a second disease identical or similar to the first disease of the patient.

5. The medical information processing apparatus of claim 4, wherein the processing circuitry estimates the first transition by applying a trained model, which is trained by using training data that is a combination of input data, which is a second progress of the second disease in the clinical case, and correct answer data which is a prescription amount of the cells relating to the second disease in the clinical case, to the first progress.

6. The medical information processing apparatus of claim 1, wherein the processing circuitry predicts the first transition with respect to each of a case where the first progress is better and a case where the first progress is worse.

7. The medical information processing apparatus of claim 1, wherein the processing circuitry predicts a name of the first disease, based on a clinical case relating to a second disease identical or similar to the first disease of the patient.

8. The medical information processing apparatus of claim 7, wherein the processing circuitry estimates the name of the first disease by applying a trained model, which is trained by using training data that is a combination of input data, which is a keyword relating to the second disease in the clinical case, and correct answer data which is a name of the second disease in the clinical case, to a keyword relating to the first disease.

9. The medical information processing apparatus of claim 1, wherein the processing circuitry estimates a name of the first disease, based on resident data including at least one of a disease tendency of residents, a population and an age distribution with respect to an area where the patient resides.

10. The medical information processing apparatus of claim 1, wherein the processing circuitry outputs a prescription order to manufacture an amount of the cells, which corresponds to the necessary amount of the cells at the future time point, to an institution that manufactures the cells.

11. The medical information processing apparatus of claim 1, wherein the first progress includes information relating to at least one of a diseased part and a symptom of the patient, which are involved in a progression of the first disease.

12. A medical information processing system comprising a medical information processing apparatus and a display device, wherein
the medical information processing apparatus comprises processing circuitry configured to:
predict a first progress relating to a first disease that a patient contracts or may contract;
specify a cell necessary for treating the first disease of the patient;
predict a first transition relating to a necessary amount of the cells, based on the first progress;
predict a second transition relating to a supply amount of the cells, based on information relating to a status of manufacture of the cells;
determine, based on the first transition and the second transition, a future time point when the necessary amount of the cells and the supply amount of the cells coincide, or when the supply amount of the cells is greater than the necessary amount of the cells; and
output, as a processing result, information relating to at least one of the first progress, the cell, the future time point, and the necessary amount of the cells at the future time point, and
the display device is configured to display the processing result that the medical information processing apparatus outputs.
